(19) United States
(12) Patent Application Publication
(10) Pub. No.: US 2021/0311069 A1
(43) Pub. Date: Oct. 7, 2021

(54) Isotopically-encoded nanoparticles for multimodal high-order multiplexed detection and imaging

(71) Applicant: The Board of Trustees of the Leland Stanford Junior University, Stanford, CA (US)

(72) Inventors: Stefan Harmsen, Menlo Park, CA (US); Ahmet F. Coskun, San Jose, CA (US)

(21) Appl. No.: 17/266,891

(22) PCT Filed: Aug. 13, 2019
(86) PCT No.: PCT/US2019/046346

§ 371 Date: Feb. 8, 2021

Related U.S. Application Data
(60) Provisional application No. 62/720,660, filed on Aug. 21, 2018.

(51) Int. Cl. G01N 33/58; G16B 40/00

(52) U.S. Cl. CPC G01N 33/587; G16B 40/00

(57) ABSTRACT

A system of barcoding isotopically encoded particles in combination with elemental analyses and imaging that includes a particulate matrix, at least one isotope label contained in the particulate matrix, where the isotope label operates as i) an elemental identifier, ii) a mass identifier, or iii) an elemental identifier and a mass identifier, where the matrix operates as multi-digit particulate barcodes, at least i) a mass-based imager, ii) an elemental analyzer, iii) or the mass-based imager and the elemental analyzer, and a debarcoding algorithm and an automated machine learning analysis algorithm programmed on a computer to computational extract the multi-digit particulate barcodes for quantification of spatial nanotag distributions in ion beam imaging areas.

### FIELD OF THE INVENTION

The present invention relates generally to biomedical imaging. More particularly, the invention relates to a system of barcoding isotopically encoded particles in combination with elemental analyses and imaging.

### BACKGROUND OF THE INVENTION

Spatial analysis of biological systems facilitates understanding of health and diseases up to the single-cell or even subcellular level. Multiparameter mapping of molecular constituents in cells and tissues has been implemented using methods based on fluorescence spectroscopy and mass spectrometry. To overcome the color limitations of microscopy, barcoded imaging of RNA labels has been used to enable spatially resolved and multiplexed genomics measurements. Imaging of mass labels allows simultaneous monitoring of up to 36 protein markers in cells using mass-labeled antibodies in combination with multiplexed ion beam imaging (MIBI) or imaging mass cytometry (IMC); however, these high-resolution analyses using secondary ion beam mass spectrometry (SIMS) methods are limited to technically available mass channels.

Gallium, helium, oxygen, or argon ion beams have been used for SIMS imaging. Oxygen primary beams are the most widely employed ion beams in commercial platforms for MIBI (IonPath®) and IMC (CyTOF®). Oxygen primary ion beams have high sensitivity and spatial resolution of 260 nm to 500 nm for alkali- and lanthanide isotopes, and for these methods antibodies are conjugated to metal-chelated polymers. Cesium ion beams offer much higher spatial resolution (i.e., 50 nm) than oxygen ion beams, and thus enable subcellular imaging or nanoscopy. However, unlike the oxygen primary ion beams, cesium ion beams have low sensitivity for lanthanides and much higher sensitivity for halogens, chalcogens, pnictogens, and metalloids. The labeling chemistry for these atoms is more difficult than the metal-chelation of lanthanides or transition-metal isotopes. This currently limits the application of mass-labeled targeting agents such as antibodies and peptides in nanoscopic molecular imaging methods with a cesium ion beam. Moreover, the elements detected in a cesium primary ion beam (e.g., Si, S, F, Cl, Br, I, Se, and Te) typically have a small number of isotopes, of which several are abundant in biological tissues. Thus, the application of such isotopes as mass labels for multiplexed ion beam imaging-based interrogation of biological samples using a cesium ion beam is highly restricted.

High-dimensional profiling of markers and analytes using approaches such as barcoded fluorescent imaging with repeated labeling and mass cytometry has allowed visualization of biological processes at the single-cell level and/or subcellular level. However, there are limitations of sensitivity and mass-channel capacity.

What is needed is a nano-barcoding platform for multiplexed ion beam imaging (MIBI) using secondary ion beam spectrometry that utilizes fabricated isotopically encoded nanotags.

### SUMMARY OF THE INVENTION

To address the needs in the art, a system of barcoding isotopically encoded particles in combination with elemental analyses and imaging that includes a particulate matrix, at least one isotope label contained in the particulate matrix, where the isotope label operates as i) an elemental identifier, ii) a mass identifier, or iii) an elemental identifier and a mass identifier, where the matrix operates as multi-digit particulate barcodes, at least i) a mass-based imager, ii) an elemental analyzer, iii) or the mass-based imager and the elemental analyzer, and a debarcoding algorithm and an automated machine learning analysis algorithm programmed on a computer to computational extract the multi-digit particulate barcodes for quantification of spatial nanotag distributions in mass imaging or elemental analyses areas.

According to one aspect of the invention, the particulate matrix includes a metal(loid) chalcogen such as a metalloid oxide (i.e., silica), a metal oxide (i.e., titanium oxide, tantalum oxide), a soft nanoparticle, a liposome, a micelle, or a lipid nanoparticle, or a combination thereof.

According to another aspect of the invention, the multi-digit nanoparticle-based barcodes include a combinatorial incorporation of an isotope into the (silica) nanoparticle matrix. In one aspect the isotopes can include halogen, chalcogen, pnictogen, such as ²H, ¹⁵N, ¹⁹F, ^{79/81}Br, or ¹²⁷I, or metal isotopes. Here, the isotopically enriched molecular scaffold for the ²H comprises N-ethyl-d5-maleimide or any other deuterium-containing scaffold or combination thereof. Further, the isotopically enriched molecular scaffold for the ¹⁵N comprises L-arginine-¹⁵N₄ or any other ¹⁵N-containing scaffold or combination thereof. Additionally, the isotopically enriched molecular scaffold for the ¹⁹F comprises trimethoxy(3,3,3-trifluoropropyl)-silane or any other ¹⁹F-containing scaffold or combination thereof. Still further, the isotopically enriched molecular scaffold for the ^{79/81}Br comprises eosin-maleimide or any other ^{79/81}Br-containing scaffold or combination thereof. In addition, the isotopically enriched molecular scaffold for the ¹²⁷I comprises L-thyroxine or any other ¹²⁷I-containing scaffold or combination thereof.

In another aspect of the invention, a modified Stöber reaction is used to produce the silica nanoparticles having diameters in a range of 90 nm to 110 nm, where the modified Stöber reaction includes a mixture of 100-nm silica nanoparticles comprising 0.7% (v/v) NH₃, 4% (v/v) of the silica precursor tetraethyl orthosilicate, and 0.31% (v/v) 3-mercaptopropyltrimethoxysilane (MPTMS) in 91% (v/v) aqueous isopropanol.

In a further aspect of the invention, N-ethyl-d5-maleimide and eosin-maleimide were reacted with 3-mercaptopropyltrimethoxysilane (MPTMS) in dimethylsulfoxide (DMSO) under ambient conditions before the metalloid oxide silica nanoparticle were synthesized, where L-thyroxine was conjugated to the MPTMS using a heterobifunctional linker succinimidyl 4-N-maleimidomethyl-1-cyclohexane-carboxylate (SMCC) in a 1:1.1:1 ratio in DMSO for uniform and covalent incorporation within the silica nanoparticle matrix.

According to one aspect of the invention, trimethoxy(3,3,3-trifluoropropyl)-silane and silane appended N-ethyl-d5-maleimide, eosin-maleimide and L-thyroxine are mixed into a Stöber reaction mixture in any (²H:¹⁹F:^{79/81}Br:¹²⁷I) isotope ratio to yield isotopically encoded silica nanotags.

In yet another aspect of the invention, trimethoxy(3,3,3-trifluoropropyl)-silane and silane appended N-ethyl-d5-maleimide, eosin-maleimide and L-thyroxine are mixed into a Stöber reaction mixture in any 1:1:1:1 (²H:¹⁹F:^{79/81}Br:¹²⁷I) isotope ratio to yield isotopically encoded silica nanotags to yield a library of 2⁴ unique barcodes.

According to one aspect of the invention, ionic metal isotopes are combinatorially mixed into a dispersion of silica nanoparticles to generate metal-based isotopically encoded silica nanotags.

In another aspect of the invention, the mass-based imaging platform includes multiplexed ion-beam imaging, or mass cytometry.

In one aspect of the invention, the elemental analysis platform includes X-ray fluorescence, energy dispersive X-ray spectroscopy, and laser induced breakdown spectroscopy.

In yet another aspect of the invention, a mixture of the isotopically encoded nanotags are applied to a substrate gold-coated silicon substrate for use in the MIBI.

### BRIEF DESCRIPTION OF THE DRAWINGS

FIGS. 1A-1C show (1A) A mixture of isotopically encoded nanotags on a gold-coated silicon substrate is raster-scanned using a cesium ion beam. Next, secondary elemental ions are analyzed using SIMS and spatially deconvoluted using debarcoding algorithms to provide quantitative information on the spatial distribution of the individual nanotags. (1B) A modified Stöber reaction that involves the addition of isotopically labeled silanes in the presence of tetraethyl orthosilicate (TEOS) and NH₄OH in an aqueous isopropanol (IPA) solution was used to synthesize 100-nm isotopically encoded isotopically encoded silica nanoparticles. The four-digit barcodes are based on labeling of silica nanoparticles with ²H, ¹⁹F, ^{79/81}Br, ¹²⁷I, or combinations thereof. (1C) Molecular structures of the isotopically labeled silanes. ²H—, ^{79/81}Br—, and ¹²⁷I-containing molecules were appended to the thiol-containing MPTMS either directly via straightforward maleimide chemistry in the case of the ²H— and ^{79/81}Br-containing scaffolds or using the heterobifunctional linker SMCC for the ¹²⁷I-containing molecular scaffold L-thyroxine, according to the current invention.

FIG. 1D shows isotopically-encoded soft nanotags, where a liposome can be isotopically encoded by using isotopically-tagged lipids (see example for a deuterium-labeled lipid). Furthermore, the aqueous compartment (center) can be loaded with isotonic solutions of ((non-)radioactive)/(non-)metal ions as well as reporters (fluorophores, iron oxides) etc. or combinations thereof, according to the current invention.

FIG. 1E shows soft nanoparticles, where micelles are labeled with (hypertonic) sodium fluoride or potassium iodide solutions encapsulated within the micellar matrix and analyzed using MIBI.

FIG. 1F shows Hydrodynamic diameters of isotopically encoded silica nanotags. The hydrodynamic diameters of each preparation of nanotags was measured in water using nanoparticle tracking analysis, according to the current invention.

FIG. 2A shows MIBI of isotopically encoded nanotags, where isotopically encoded nanotags were imaged using MIBI. The MIBI data are generated by raster scanning of the samples with a cesium ion beam followed by secondary ion mass spectrometry. All silica-based nanotags were imaged in the ²⁸Si mass channel. MIBI data are presented for mass channels of ²H, ¹⁹F, ^{79/81}Br, and ¹²⁷I. The four-digit nanotags were assigned based on signal in the mass channel (‘1’) or no signal above background (‘0’). The secondary electron (SE) image reflects total electrons ejected from the sample. Scale bars, 1 μm, according to the current invention.

FIG. 2B shows intraparticle isotope distribution, where sequential cesium ion beam scans of the ¹⁹F-labeled nanotags (0100) over 50-slices (Z, 5-10 nm step size) show uniform three-dimensional distribution of ¹⁹F throughout the nanotag matrix. Scale bars, 500 nm, according to the current invention.

FIGS. 3A-3H show combinatorial nanotag barcoding with four isotope mass channels (3A-3B) Nanotags were imaged on a gold-coated silicon substrate. Shown are (3A) secondary electron beam image (SE) and b) merged MIBI image from mass channels corresponding to ²⁸Si, ¹⁹F, ^{79/81}Br, and ¹²⁷I isotopes. Numbered arrows indicate nanotags with different barcodes. Scale bars, 2 μm. (3C) Higher magnification images of nanotags numbered in panels a and b. As expected, all but one barcode (i.e., 011) were detected. Scale bars, 100 nm. (3D) Histogram displaying the quantification per barcode of the 99 isotopically encoded nanotags detected in the field of view. (3E) Number of 001 and 101 nanotags detected using manual counting and machine learning. (3F) Principal component (PC) analysis of distributions of nanotags with barcodes color-coded as indicated. (3G) t-SNE plot of nanotag subtypes provide accurate classification, according to the current invention.

FIG. 4 shows additional mass labels used for isotope encoding of nanotags. Molecular structures of the scaffolds that were used to produce covalently incorporated ¹⁵N-enriched nanotags, covalently incorporated natural-abundance Se nanotags (abundances of isotopes ⁷⁶Se, ⁷⁷Se, ⁷⁸Se, and ⁸⁰Se are 9.2%, 7.6%, 23.7%, 49.8%, respectively), and non-covalently incorporated, natural abundance Te nanotags (abundances of ¹²²Te, ¹²⁴Te, ¹²⁵Te, ¹²⁸Te and ¹³⁰Te isotopes are 2.6%, 4.7%, 7.1%, 18.8%, 31.7%, and 34.1%, respectively), according to the current invention.

FIG. 5 shows evaluation of ¹⁵N as an isotope label. Indicated channels for an ¹⁵N, ¹⁹F, ^{79/81}Br and ¹²⁷I-encoded nanotag with 1111 barcode demonstrated that all isotopes signal overlapped between the channels, according to the current invention.

FIG. 6 shows MIBI results, where the top row shows MIBI results for nanotags that were covalently encoded with natural abundant Se. Since ⁸⁰Se is the most abundant isotope (˜49.8%) we selected the 80-Da mass channel to image ⁸⁰Se. Signal in ²⁸Si and ⁸⁰Se overlap indicating that the selenium signal is associated with the silica nanoparticles. No significant signal was detected in the 130-Da mass channel, where ¹³⁰Te would be detected. The bottom row shows the MIBI results for nanotags that were non-covalently labeled with Te. Te was imaged in the 130-Da channel as ¹³⁰Te is the most abundant isotope (at 34%), according to the current invention.

FIGS. 7A-7B show stoichiometric labeling with distinct concentration levels of ⁸¹Br isotopes. (7A) Nanotag images for 1× (top row) and 2x (bottom row) concentrations of ⁸¹Br encoded in nanotags (see Table 51 for clarification on how different molar ratios of the isotopes were incorporated). Scale bars, 1 μm. (7B) Bar plot of the median distribution of ⁸¹Br/²⁸Si ratio for Br-1× (n=9 nanotags) and Br-2× (n=29 nanotags). Individual nanotags were digitally segmented, isolated, and quantified for total ion signal in ⁸¹Br and ²⁸Si signals per nanotag. Student's t-test, *** p<6.45×10⁻⁵, according to the current invention.

FIGS. 8A-8B show stochiometric barcoding with ratios of ¹⁹F to ^{79/81}Br isotopes. (8A) The top row shows MIBI images of a nanotag mixture of ¹⁹F, ^{79/81}Br, and ¹²⁷I at a ratio of 1:1:1, and the bottom images are of a mixture at a ratio of 0.5:2:1. Scale bars, 1 μm. (8B) Ratio of indicated isotopes to ²⁸Si for the 1:1:1 mixture (n=8 nanotags) and for the 0.5:2:1 mixture (n=58 nanotags), according to the current invention.

FIGS. 9A-9D show machine-learning analysis of nanotag images. (9A) Absolute number of nanotags detected using manual counting and machine learning for nanotag mixtures of ¹⁹F, ^{79/81}Br, and ¹²⁷I at ratios of 1:1:1 (n=8) and 0.5:2:1 (n=58). The machine learning pipeline was based on support vector machine (SVM)-based training and prediction. (9B-9D) Classification results of SVM for b)^{79/81}Br/²⁸Si vs. ¹⁹F/²⁸Si, c) ¹²⁷I/²⁸Si vs. ¹⁹F/²⁸Si, and d) ¹²⁷I/²⁸Si vs. ^{79/81}Br/²⁸Si. The corresponding 1:1:1 and 0.5:2:1 barcoded nanotags are shown, which were separated by a Gaussian kernel on the decision surface. Individual nanotags segmented, digitally quantified for total ion signal per channel as a feature vector. Nanotags were automatically classified based on ratiometric isotope barcoding scheme. The selected fields denote classification region for each nanotag, where the outlines indicate the expected region for 1:1:1-ratio barcoded nanotags, and the 0.5:2:1-ratio barcoded nanotags, according to the current invention.

### DETAILED DESCRIPTION

The current invention provides a system of barcoding isotopically encoded particles in combination with elemental analyses and imaging that includes a particulate matrix, at least one isotope label contained in the particulate matrix, where the isotope label operates as i) an elemental identifier, ii) a mass identifier, or iii) an elemental identifier and a mass identifier, where the matrix operates as multi-digit particulate barcodes, at least i) a mass-based imager, ii) an elemental analyzer, iii) or the mass-based imager and the elemental analyzer, and a debarcoding algorithm and an automated machine learning analysis algorithm programmed on a computer to computational extract the multi-digit particulate barcodes for quantification of spatial nanotag distributions in ion beam imaging areas.

According to one aspect of the invention, the particulate matrix includes a metal(loid) chalcogen such as a metalloid oxide (i.e., silica), a metal oxide (i.e., titanium oxide, tantalum oxide, etc.), a soft nanoparticle, a liposome, a micelle, or a lipid nanoparticle, or a combination thereof.

According to embodiments of the invention, the mass-based imaging platform includes multiplexed ion-beam imaging, or mass cytometry. In further embodiments of the invention, the elemental analysis platform includes X-ray fluorescence, energy dispersive X-ray spectroscopy, and laser induced breakdown spectroscopy.

One embodiment includes a nano-barcoding platform for multiplexed ion beam imaging (MIBI) using secondary ion beam spectrometry that utilizes fabricated isotopically encoded nanotags. In one embodiment, the invention uses combinatorial isotope distributions in 100-nm-sized nanotags to expand the labeling palette to overcome the spectral bounds of mass channels. In an exemplary embodiment, a four-digit (i.e., 0001 to 1111) barcoding scheme is provided to detect 16 variants of ²H, ¹⁹F, ^{79/81}Br and ¹²⁷I elemental barcode sets that are encoded in silica nanoparticle matrices. A computational debarcoding method and an automated machine learning analysis approach are provided to extract barcodes for accurate quantification of spatial nanotag distributions in large ion beam imaging areas up to 0.6 mm². Isotopically encoded nanotags should boost the performance of mass imaging platforms such as MIBI and other elemental-based bioimaging approaches.

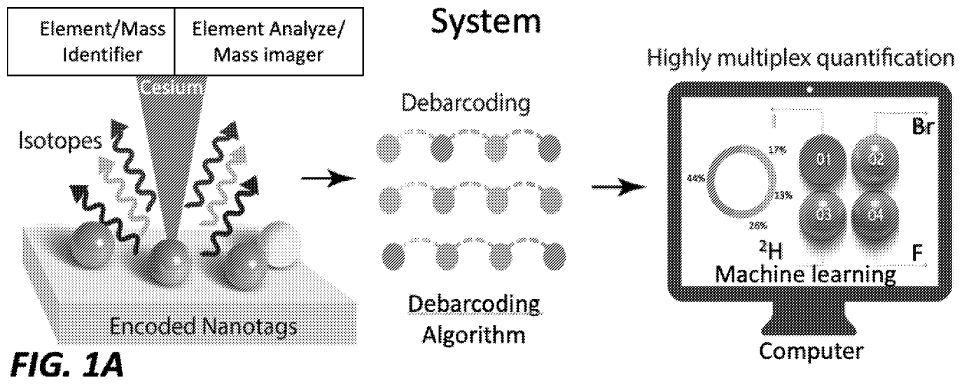

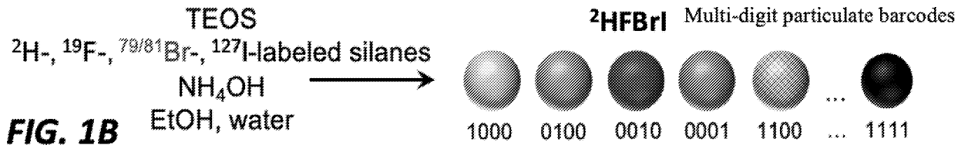

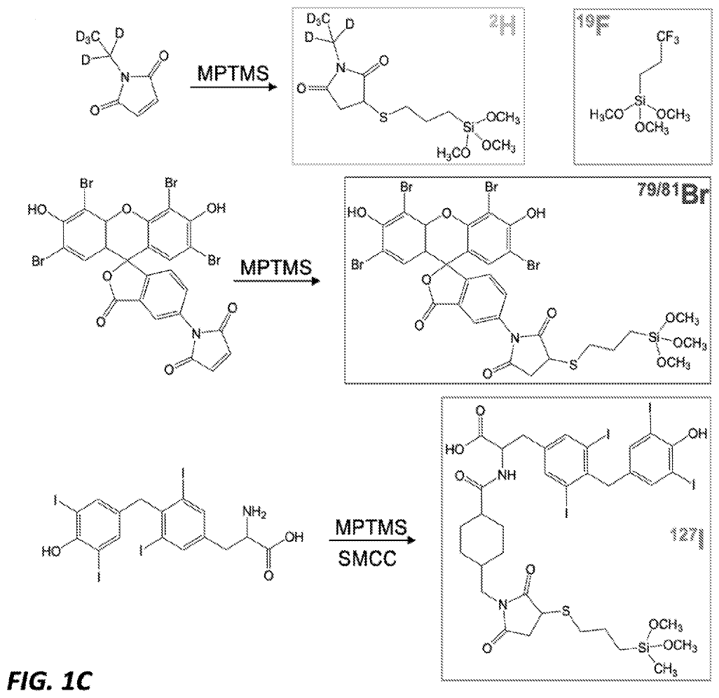

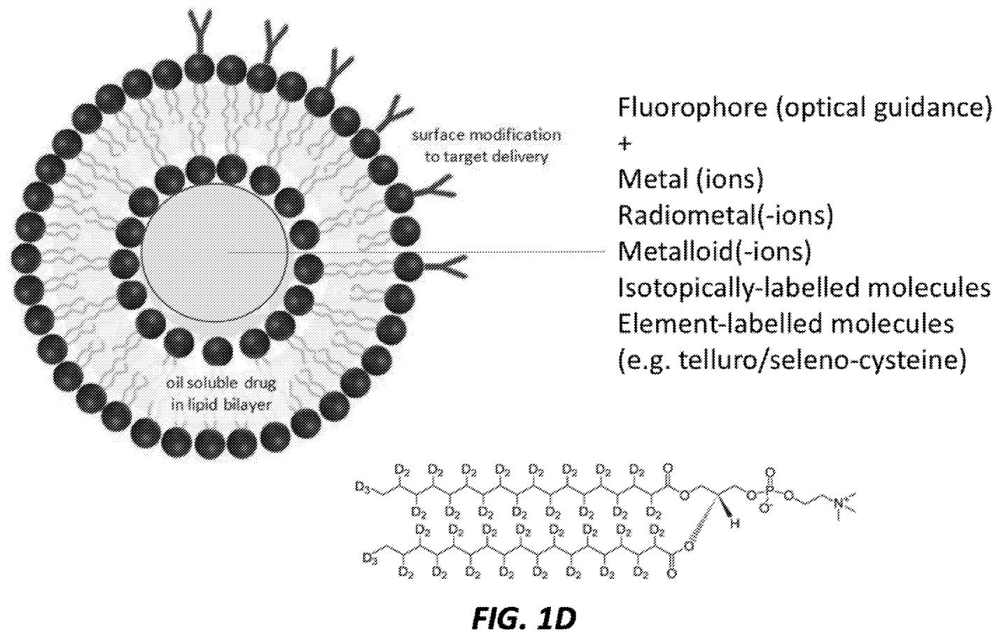

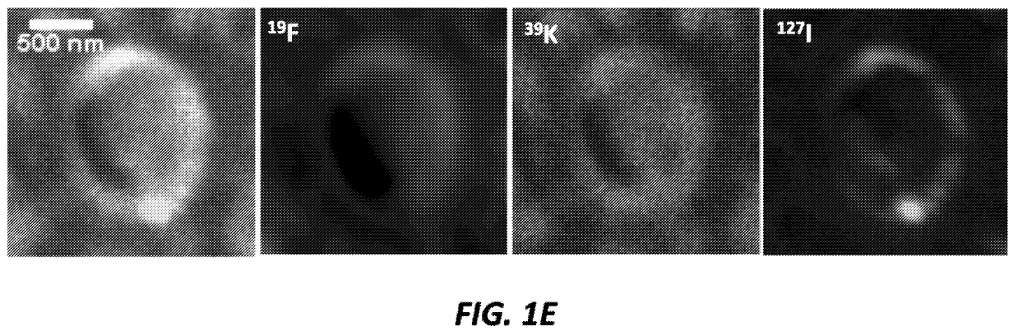

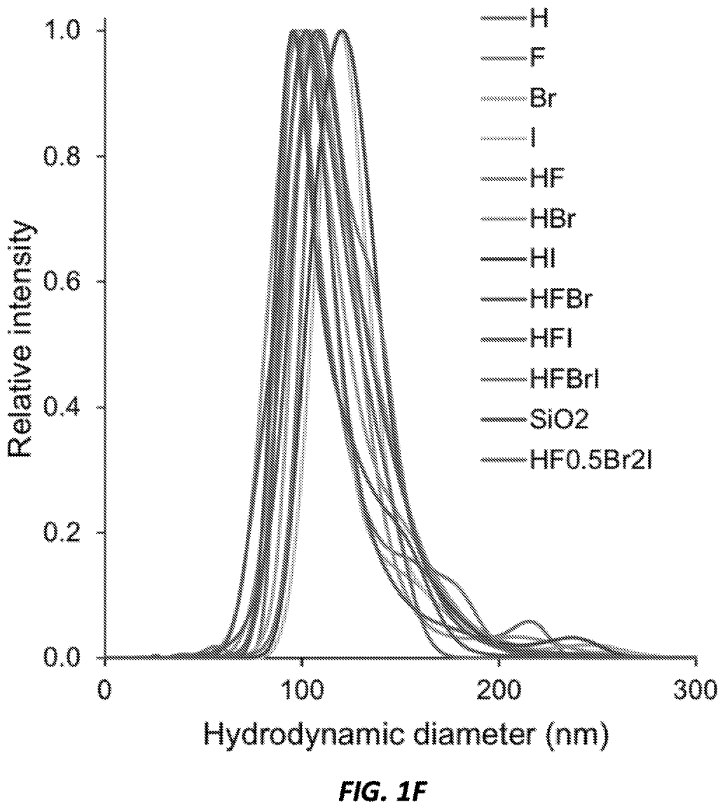

According to the current invention, a nano-barcoding platform that is based on metal and metalloid oxide nanoparticles is provided. The method relies on combinatorial incorporation of halogen, chalcogen, and pnictogen isotopes of low biological abundance (i.e., ²H, ¹⁵N, ¹⁹F, ^{79/81}Br, and ¹²⁷I) into a silica nanoparticle matrix to produce isotopically encoded nanotags (FIG. 1A). Here, the isotopically enriched molecular scaffold for the ²H comprises N-ethyl-d5-maleimide. Further, the isotopically enriched molecular scaffold for the ¹⁵N comprises L-arginine-¹⁵N₄. Additionally, the isotopically enriched molecular scaffold for the ¹⁹F comprises trimethoxy(3,3,3-trifluoropropyl)-silane. Still further, the isotopically enriched molecular scaffold for the ^{79/81}Br comprises eosin-maleimide. In addition, the isotopically enriched molecular scaffold for the ¹²⁷I comprises L-thyroxine. The metalloid oxide silica is provided as the matrix for the nanoparticle-based barcodes, because silica precursors and methods for synthesis of silica nanoparticles of controlled sizes are available and silica surface modifications to enable antibody conjugation are understood. A modified Stöber reaction is used to produce silica nanoparticles with diameters of in a range of 90 nm to 110 nm. According to one embodiment, a reaction mixture for the synthesis of 100-nm silica nanoparticles contains 0.7% (v/v) NH₃, 4% (v/v) of the silica precursor tetraethyl orthosilicate, and 0.31% (v/v) 3-mercaptopropyltrimethoxysilane (MPTMS) in 91% (v/v) aqueous isopropanol. After a reaction time of 30 minutes under ambient conditions, the 100-nm silica nanoparticles were collected using centrifugation (5 min at 10,000 g) and washed with 100% ethanol to afford 100-nm silica nanoparticles.

In one aspect of the invention, isotopically encoded silica nanotags are provided by linking silane-appended isotopically enriched molecular scaffolds to yield four-digit barcodes (FIG. 1B). N-ethyl-d5-maleimide, trimethoxy(3,3,3-trifluoropropyl)-silane, eosin-maleimide, and L-thyroxine, are provided as the molecular scaffolds for ²H, ¹⁹F, ^{79/81}Br, and ¹²⁷I, respectively. Due to the low natural abundance of ²H, a ²H-enriched scaffold is used. The other scaffolds were based on isotopes of high natural abundance: Natural abundances of ¹⁹F, ^{79/81}Br, and ¹²⁷I are 100%, 51/49%, and 100%, respectively. To enable uniform, covalent incorporation within the silica nanoparticle matrix, N-ethyl-d5-maleimide and eosin-maleimide were first reacted overnight with MPTMS in dimethylsulfoxide (DMSO) under ambient conditions and then silica nanoparticles were synthesized. L-thyroxine was conjugated to MPTMS using the heterobifunctional linker succinimidyl 4-N-maleimidomethyl-1-cyclohexane-carboxylate (SMCC) in a 1:1.1:1 ratio in DMSO (FIG. 1C). Silane-appended eosin-maleimide and L-thyroxine derivatives were used without further purification. The concentrations of silane-appended isotopically enriched scaffolds were normalized based on the isotope abundance in the scaffold and were added independently or as a combination as a fraction of the 0.31% of the MPTMS volume fraction (Table 1). As shown using nanoparticle tracking analysis, the average hydrodynamic diameter of the nanotags was 105.5±8.1 nm with a coefficient of variance of 7.6% between different nano-barcodes (FIG. 1D).

TABLE 1
Isotopic encoding of silica nanotags based on normalized addition of the
individual isotope scaffolds.

| # | Isotope | Barcode | ²H₅ | ¹⁹F₃ | ^{79/81}Br₄ | ¹²⁷I₄ | MPTMS | total |
|---|---|---|---|---|---|---|---|---|
| 0 | — | 0000 | | | | | 0.31% | 0.31% |
| 1 | H | 1000 | 0.060% | | | | 0.25% | 0.31% |
| 2 | F | 0100 | | 0.10% | | | 0.21% | 0.31% |
| 8 | Br | 0010 | | | 0.075% | | 0.24% | 0.31% |
| 4 | I | 0001 | | | | 0.075% | 0.24% | 0.31% |
| 5 | H, F | 1100 | 0.060% | 0.10% | | | 0.15% | 0.31% |
| 6 | H, Br | 1010 | 0.060% | | 0.075% | | 0.18% | 0.31% |
| 7 | H, I | 1001 | 0.060% | | | 0.075% | 0.18% | 0.31% |
| 8 | H, F, Br | 1110 | 0.060% | 0.10% | 0.075% | | 0.075% | 0.31% |
| 9 | H, F, I | 1101 | 0.060% | 0.10% | | 0.075% | 0.075% | 0.31% |
| 10 | H, F, Br, I | 1111 | 0.060% | 0.10% | 0.075% | 0.075% | — | 0.31% |
| | (1:1:1:1) | | | | | | | |
| 11 | H, F, Br, I | 2142 | 0.060% | 0.05% | 0.150% | 0.075% | — | 0.33% |
| | (1:0.5:2:1) | | | | | | | |

*The addition of the isotope scaffolds was normalized based on the molar isotope ratio between the scaffolds. For instance, silane-¹⁹F₃ incorporate 3 moles of ¹⁹F per 1 mole of scaffold and thus 4/3 × the molar amount of silane-¹⁹F₃ relative to eosin-maleimide, which contains 4 moles of Br per 1 mole of scaffold, was added in the reaction mixture.

FIG. 1E shows soft nanoparticles, where micelles are labeled with (hypertonic) sodium fluoride or potassium iodide solutions encapsulated within the micellar matrix and analyzed using MIBI. FIG. 1F shows Hydrodynamic diameters of isotopically encoded silica nanotags. The hydrodynamic diameters of each preparation of nanotags was measured in water using nanoparticle tracking analysis, according to the current invention.

Next, gold-coated silicon substrates are prepared with a 200-nm thick gold layer coated on a 20-nm titanium adhesion layer (substrate dimensions: 7×7 mm, Silicon Valley Microelectronics) using the Innotec E-beam metal evaporation system. Gold was selected because the mass of gold is 197 Da (100% natural abundance), which should not interfere with MIBI of the silica nanoparticles. A dispersion of isotopically encoded silica nanoparticles in ethanol (2-5 μL) was placed on the gold-coated silicon substrate and air-dried overnight before ion beam imaging.

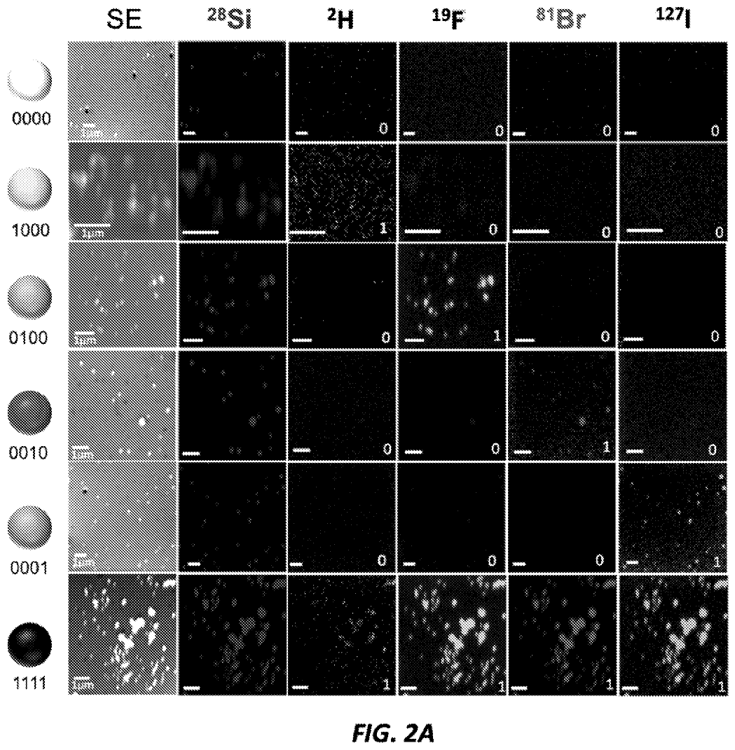

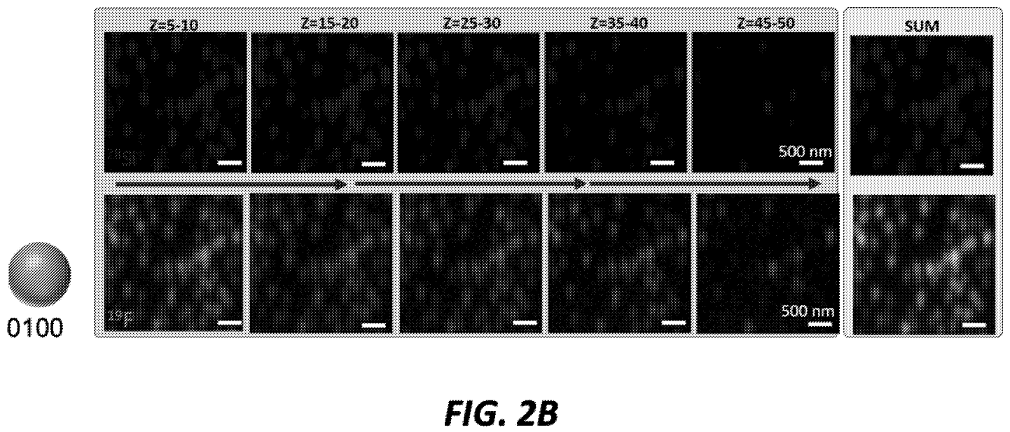

To validate the incorporation of the individual isotopes into the silica nanoparticle matrix, MIBI was performed on nanotags without isotope encoding or with single or all isotopes. Bare silica nanoparticles had positive signal only in the ²⁸Si mass channel, and no significant background was observed in the mass channels corresponding to ²H, ¹⁹F, ^{79/81}Br, or ¹²⁷I isotope labels. Since the barcoding system is based on combinatorial encoding with these four isotopes (FIG. 1B), the associated four-digit barcode for bare silica corresponds to 0000. Nanotags that were encoded with a single isotope had signal only in the mass channel of the respective isotope (FIG. 2A). The isotope distributions were analyzed within the silica nanoparticle matrix and it was found that the isotope-label for ¹⁹F-encoded nanotags closely matched ²⁸Si indicating that the labels are uniformly distributed within the silica nanoparticle matrix (FIG. 2B). MIBI of the nanotags incorporating all selected isotopes demonstrated that all mass-channels recorded positive signals, corresponding to a 1111 barcode (FIG. 2). Of note, the high mass-spectral separation that is needed for the ²H mass label identification resulted in lower sensitivity of ²H detection, relative to sensitivities of the halogen isotopes, which had smaller aperture settings. Due to the dynamic range difference (˜10-fold) between the ²H and halogen mass channels, the ²H mass channel was excluded for quantification purposes.

To demonstrate the utility of the barcoding strategy, an exemplary isotopically encoded nanotag mixture was prepared containing the three-digit (¹⁹F, ^{79/81}Br, ¹²⁷I) barcode combinations 000, 100, 010, 001, 110, 101, and 111; this is all possible combinations except for 011 because of it was not included in the prepared nanotag mixture. 5 μl of this mixture was deposited on the gold-coated silicon substrate. A large raster scan (512×512 pixels) was performed with data from 10 scans collected at a scanning speed of 5 minutes per scan using a NanoSIMS device.

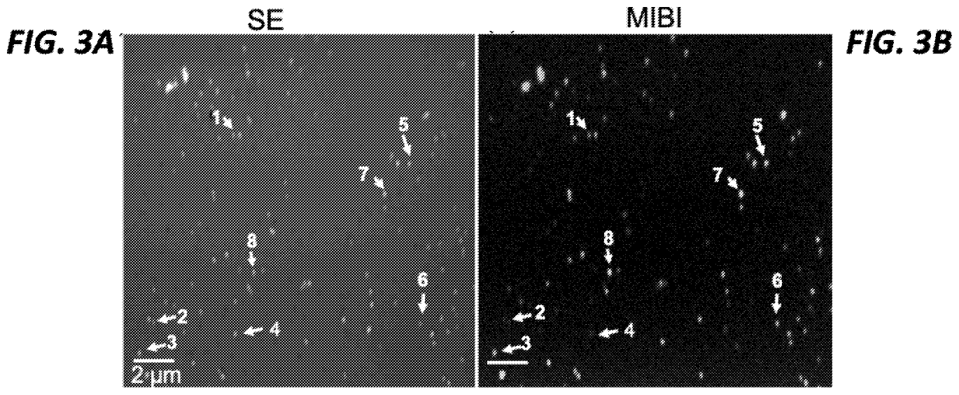

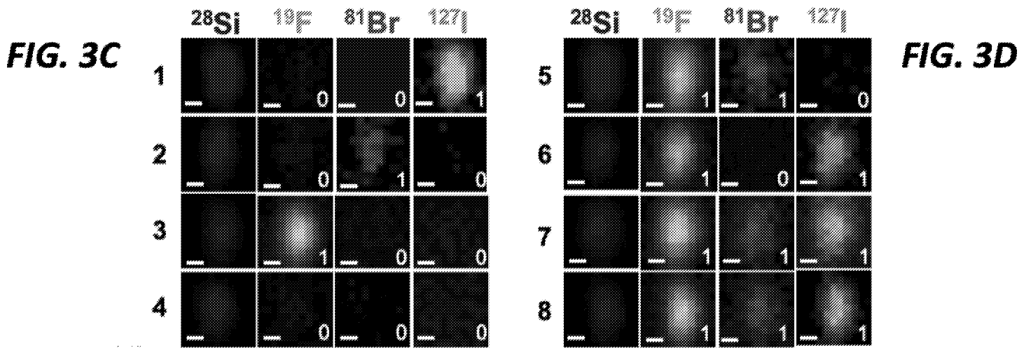

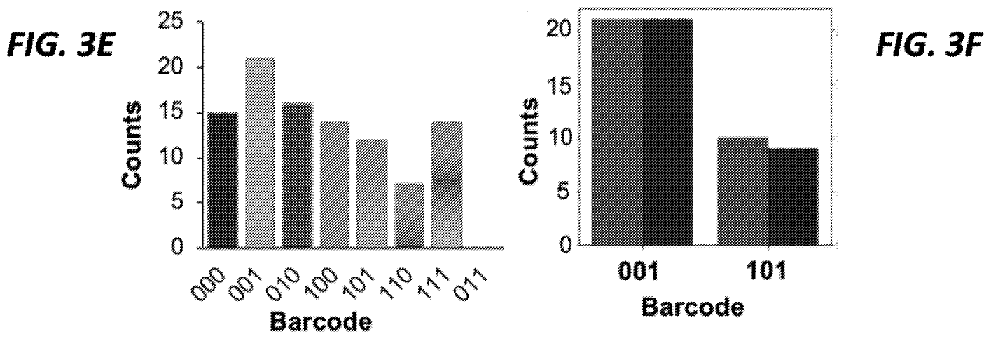

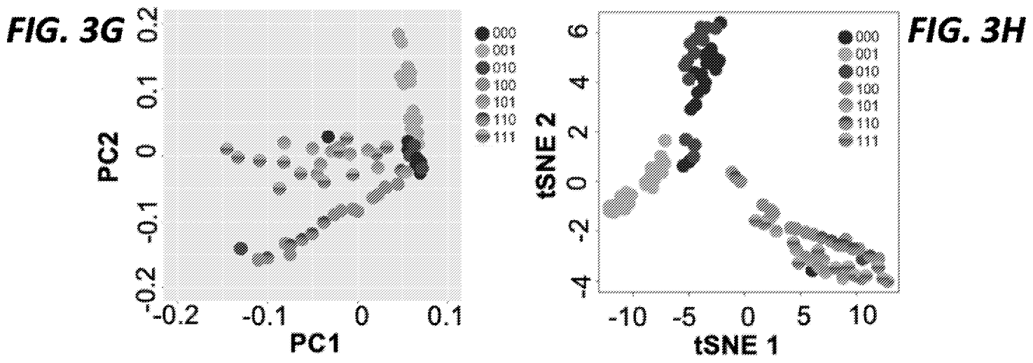

The secondary electron image showed that most nanotags were isolated (FIG. 3A), which proved ideal for digital quantification. In the merged MIBI image 99 nanotags were within the field of view when the ²⁸Si signal was used as a nanotag identifier (FIG. 3B). The barcode of each nanotag was extracted based on ¹⁹F, r, ^{79/81}Br, and ¹²⁷I mass channel signals (FIG. 3C). Detected were 15, 21, 16, 14, 12, 7, and 14 counts for the three-digit barcodes (¹⁹F, ^{79/81}Br, ¹²⁷I) 000, 001, 010, 100, 101, 110, and 111, respectively (FIG. 3D).

Barcode assignment was then automated by an unsupervised machine learning algorithm. Each isotope channel was treated as a feature vector that was used in the training and prediction. A mathematical basis for support vector machine (SVM) was used to deconvolve the barcoded nanotags. Direct digital and manual quantification and machine learning-based prediction of nanotag barcodes (e.g., 001 debarcoded from 101) provided good agreement in the bar plot (FIG. 3E). Classified barcode sets were then visualized on the principal component analysis and t-Distributed Stochastic Neighbor Embedding (t-SNE) plots (FIGS. 3F-3G), demonstrating the accurate debarcoding of the presented highly multiplexed nanotag results.

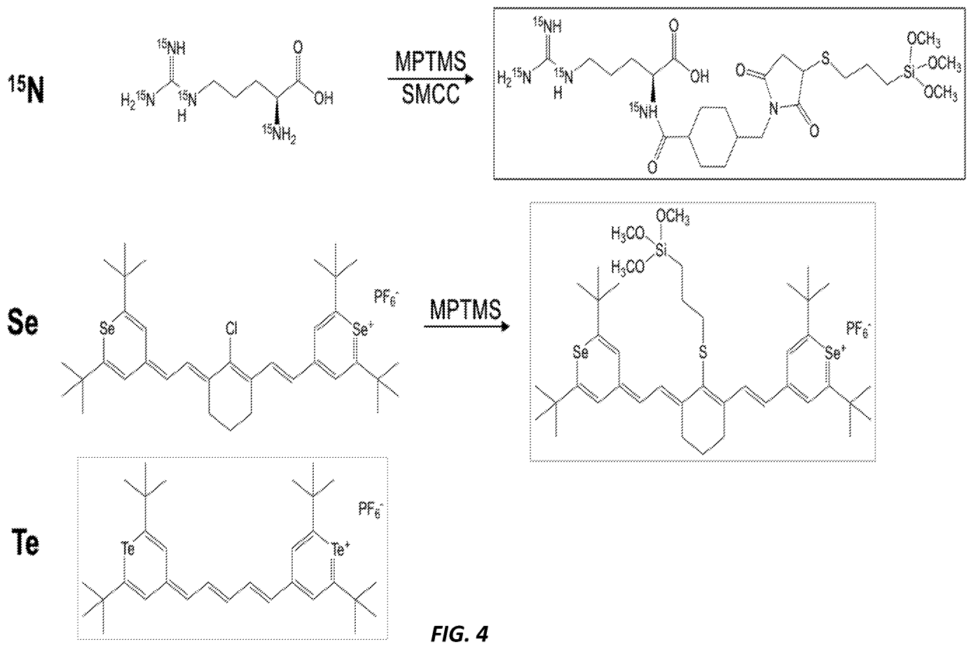

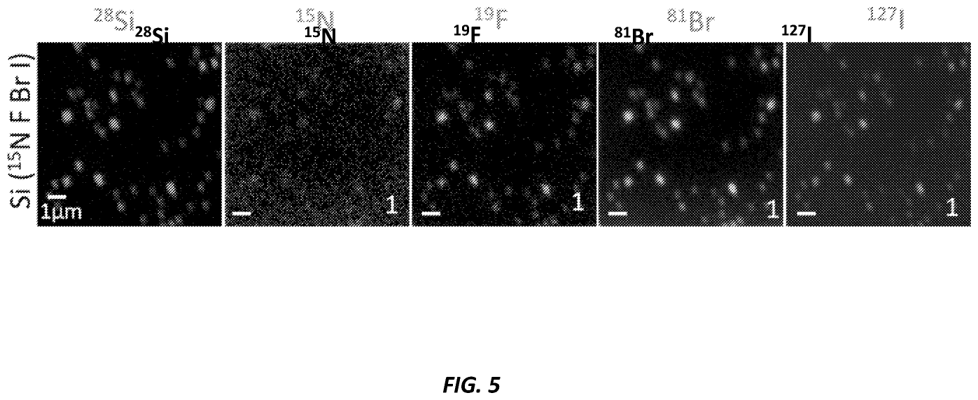

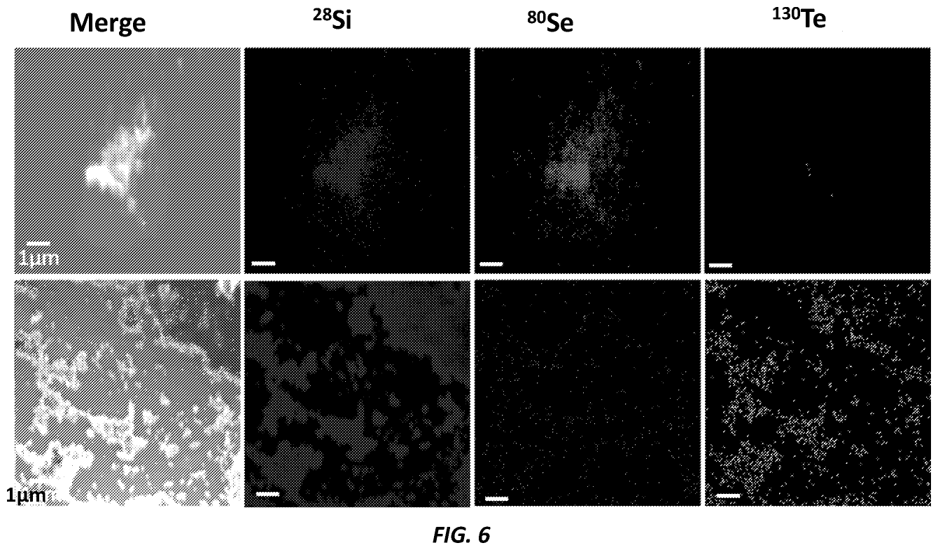

In addition to ²H, ¹⁹F, ^{79/81}Br, and ¹²⁷I mass-labels, we also explored the incorporation of ¹⁵N-enriched and natural abundance ^{76/77/78/80}Se— and ^{122/124/125/128/130}Te— containing scaffolds (FIG. 4). As a molecular scaffold for ¹⁵N, arginine-¹⁵N₄ was selected, which was conjugated to MPTMS using SMCC to enable covalent incorporation of the ¹⁵N-scaffold into the silica matrix of the nanotags. The ¹⁵N-¹⁹F-^{79/81}Br-¹²⁷I barcode (1111) was distinguished from the ¹⁹F-^{79/81}Br-¹²⁷I barcode (0111) scheme (FIG. 5). For Se— and Te— incorporation, meso-chloro-substituted selenopyrylium and telluropyrylium scaffolds were used. The meso-chloro-substituted selenopyrylium was conjugated with MPTMS by reacting it overnight in DMSO at 72° C. in a 1:1 molar ratio to enable covalent incorporation into the nanotag matrix. The telluropyrylium scaffold was used without appending to silane. Se— and Te-encoded nanotags were prepared and imaged by MIBI demonstrating that ⁸⁰Se was detectable and overlapped with specific ²⁸Si host matrices, with no spillover into the ¹³⁰Te mass channel (FIG. 6). Similarly, specific signals were observed for ¹³⁰Te with minimal ion signal in the ⁸⁰Se mass channel (FIG. 6). Compared to ⁸⁰Se, which was silane-appended, non-covalent incorporation of the Te-scaffold yielded lower signal under identical imaging conditions due to lower levels of incorporation of the tellurium scaffold into the silica nanoparticle matrix.

Since selenium and tellurium have mass overlap with bromine and iodine, respectively, neither was further explored for use in the isotopically encoded nanotags mixtures. In contrast, ²H and ¹⁵N do not have mass overlap with any of the halogens that were successfully incorporated in the isotopically encoded nanotags. Moreover, since the sensitivity of ²H and ¹⁵N mass labels is related to the aperture setting, the sensitivity of both isotopes could be improved by increasing the stochastic ratio of ²H and ¹⁵N relative to the halogens. Incorporation of ²H and ¹⁵N in addition to ¹⁹F, ^{79/81}Br, ¹²⁷I into the current set of nanotags will enable the generation of 2⁵ or 64 distinct barcodes.

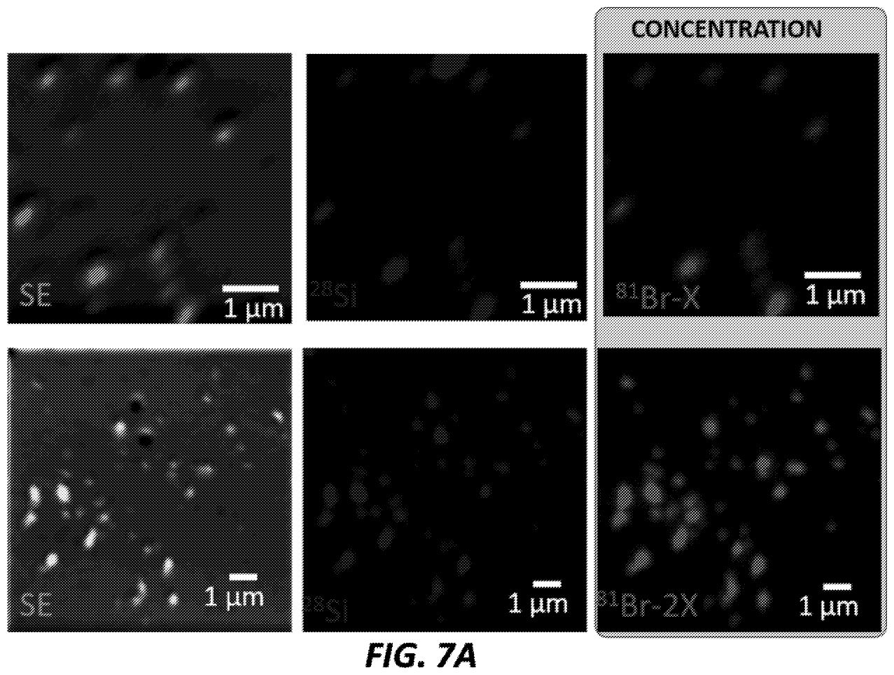

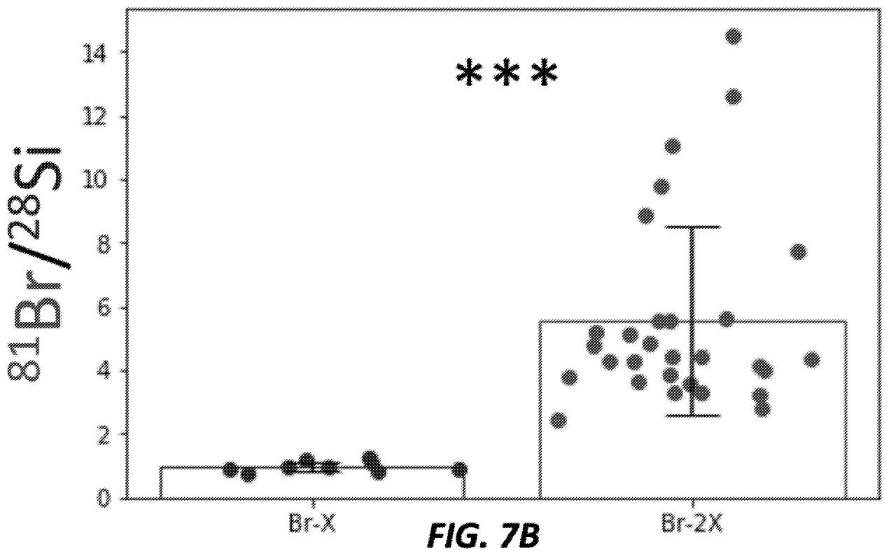

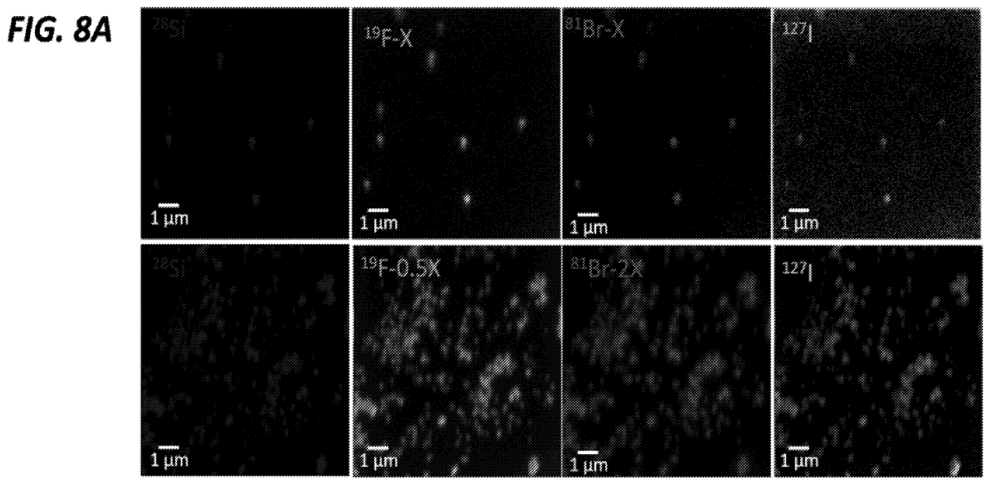

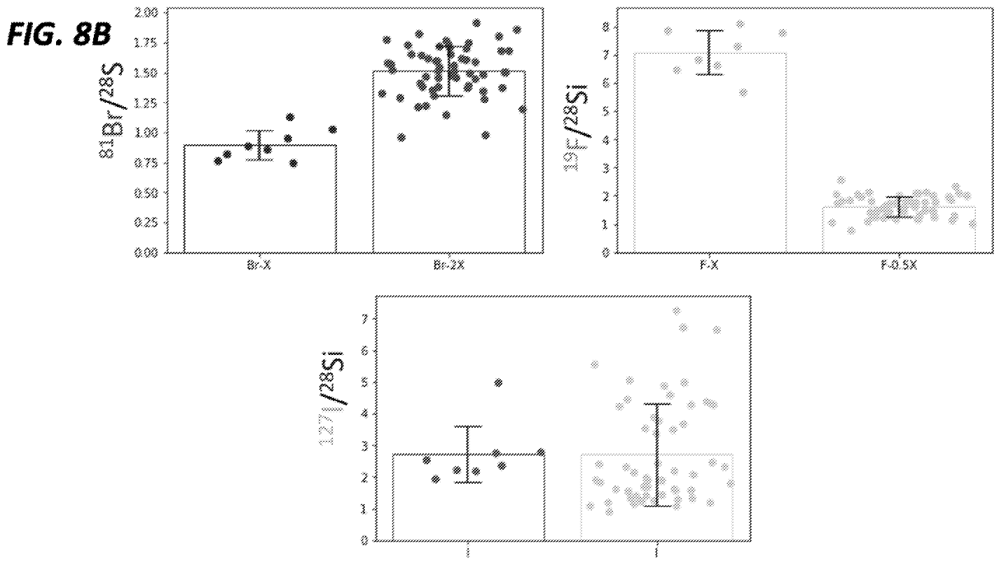

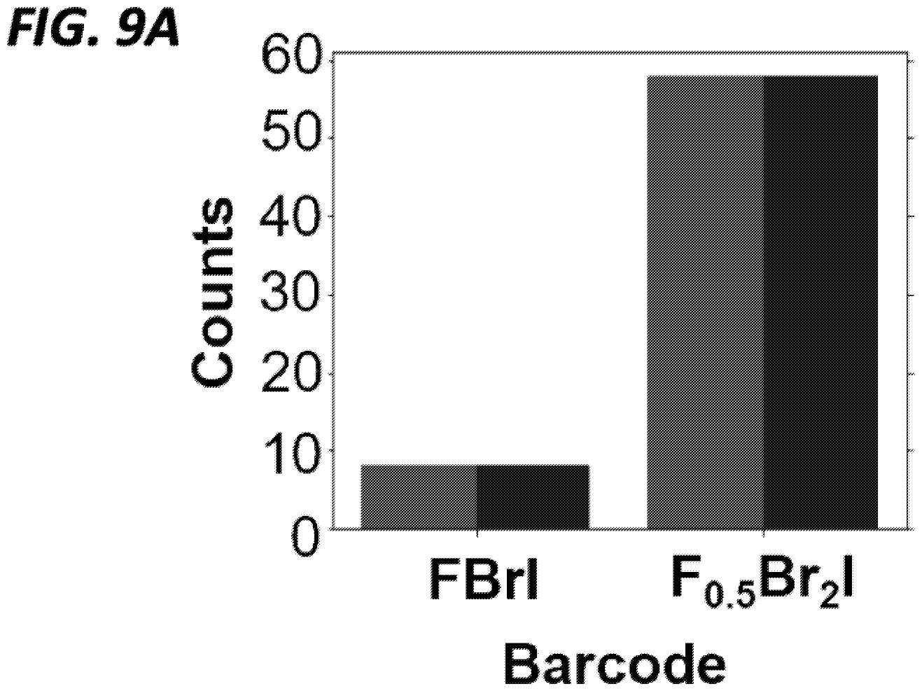

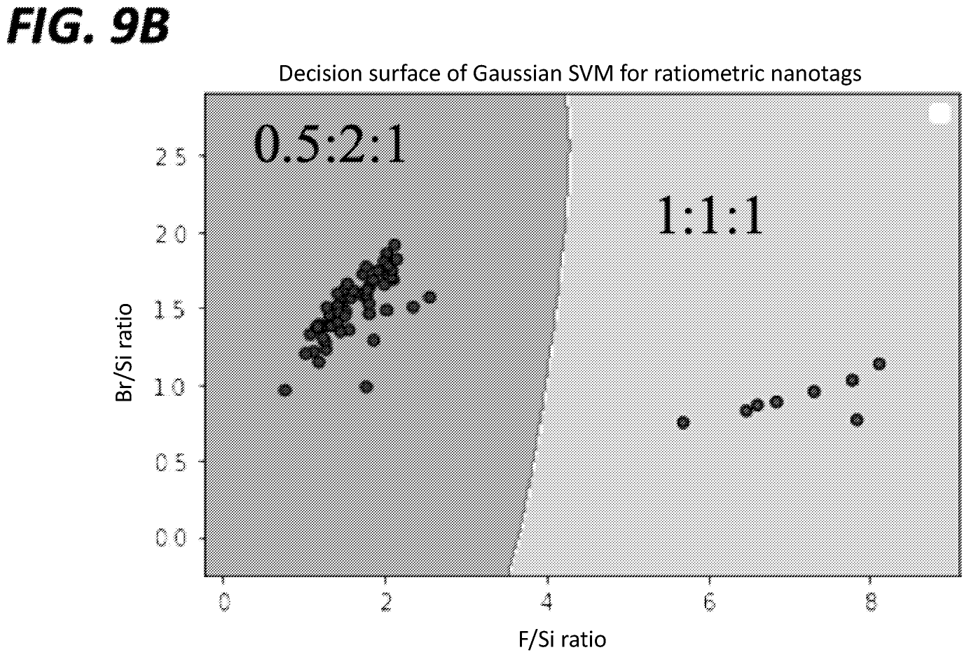

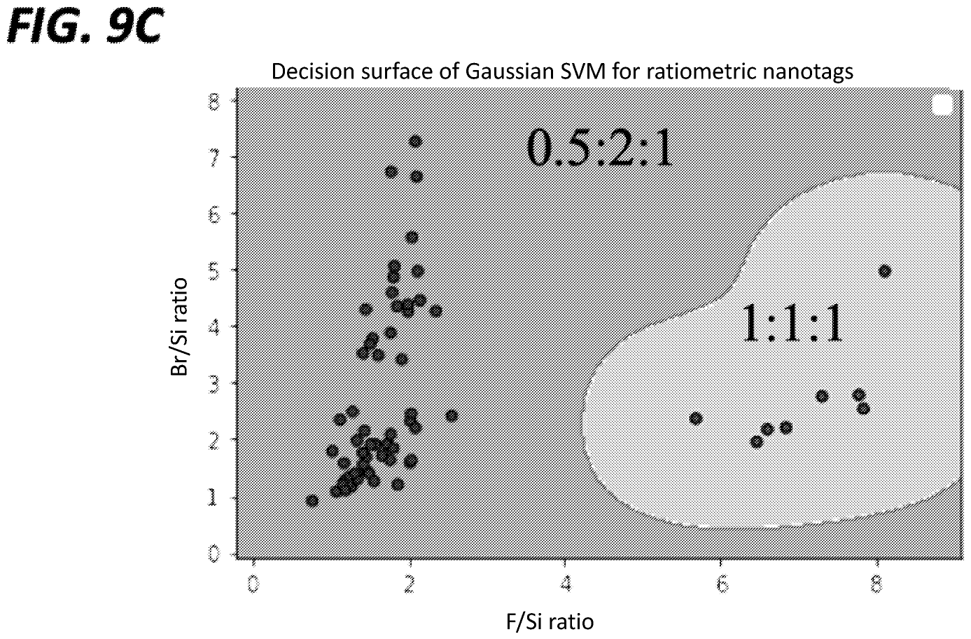

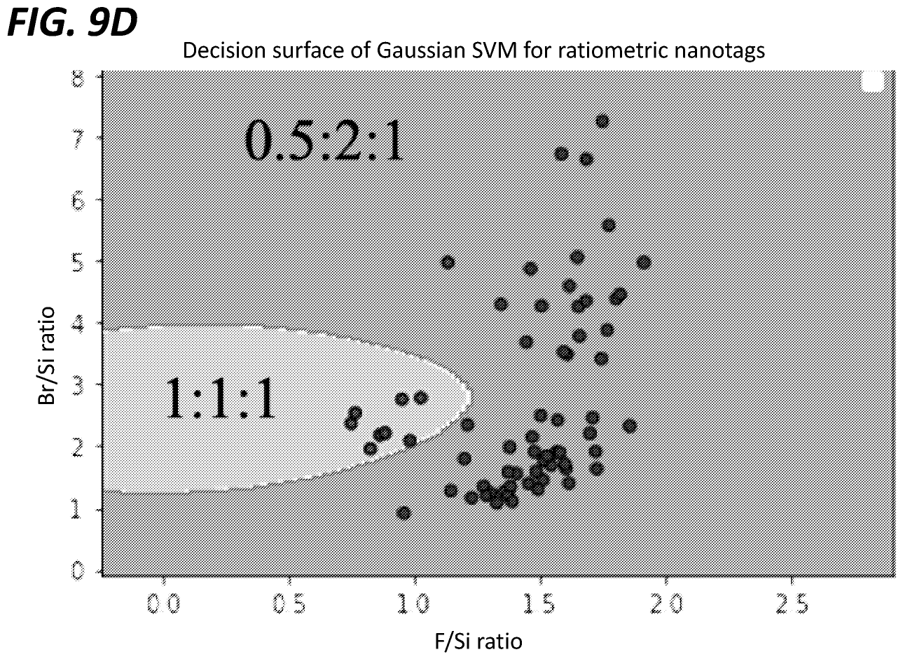

As an alternative method for expansion of the barcode library, we also explored the incorporation of different stoichiometric ratios of the halogen isotopes (Table 1, entry 11). Nanotags prepared with ¹⁹F—^{79/81}Br-¹²⁷I in a 1:1:1 stoichiometric ratio were able to be separated from those prepared in a 0.5:2:1 ratio (FIGS. 7A-7B and FIGS. 8A-8B). This was achieved by measuring the intensity of signal of each isotope relative to the signal of ²⁸Si-silica nanoparticle matrix. Machine-learning debarcoding successfully classified barcodes based on the ratiometric analysis (FIGS. 9A-9D).

In summary, isotopically encoded nanotags were synthesized that combinatorially incorporate ¹⁹F, ^{79/81}Br, and ¹²⁷I to generate a library of nanobarcodes for multiplexed analysis in nanoscopic applications using cesium ion beams. The nanotags were uniformly labeled with the isotopes. The ratios of different nanotags in mixtures were successfully determined automatically via digital analysis and a machine-learning framework. Since silica surface modification is straightforward, the nanotags can be conjugated to analyte capturing moieties such as aptamers, peptides, or antibodies to enable highly sensitive and multiplexed analyte detection or imaging.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example, the nanobarcodes can be conjugated to antibodies to enable high-level multiplexed detection of analytes during mass imaging-based histopathology or mass-cytometry. In addition, the nanobarcodes can be used to study analyte-biomolecule interactions where a subset of nanotags is labeled with the analyte and the other subset with the biomolecule of interest. By analyzing the proximity of the different nanotags using automated counting and interparticle distance measurements binders and non-binders for a specific analyte/biomolecule can be identified in tandem. Moreover, nanobarcodes can be used as anticounterfeiting labels for art, money, or any other object to establish the authenticity using mass-label or non-destructive elemental analyses (i.e. XRF) approaches.

All such variations are considered to be within the scope and spirit of the present invention as defined by the following claims and their legal equivalents.

What is claimed:

1) A system of barcoding isotopically encoded particles in combination with elemental analyses and imaging, compromising:
a) a particulate matrix;
b) at least one isotope label contained in said particulate matrix, wherein said isotope label operates as i) an elemental identifier, ii) a mass identifier, or iii) said elemental identifier and said mass identifier, wherein said matrix operates as multi-digit particulate barcodes;
c) at least i) a mass-based imager, ii) an elemental analyzer, iii) or said mass-based imager and said elemental analyzer; and
d) a debarcoding algorithm and an automated machine learning analysis algorithm programmed on a computer to computational extract said multi-digit particulate barcodes for quantification of spatial nanotag distributions in ion beam imaging areas.

2) The system according to claim 1, wherein said particulate matrix is selected from the group consisting of a metal(loid) chalcogen, a metalloid oxide, silica, titanium oxide, tantalum oxide, a soft nanoparticle, a liposome, a micelle, and a lipid nanoparticle.

3) The system according to claim 1, wherein said multi-digit nanoparticle-based barcodes comprise a combinatorial incorporation of an isotope into said silica nanoparticle matrix.

4) The system according to claim 3, wherein said isotopes are selected from the group consisting of halogen, chalcogen, pnictogen, metal isotopes, ²H, ¹⁵N, ¹⁹F, ^{79/81}Br, and ¹²⁷I.

5) The system according to claim 4, wherein said isotopically enriched molecular scaffold for said ²H comprises N-ethyl-d5-maleimide.

6) The system according to claim 4, wherein said isotopically enriched molecular scaffold for said ¹⁵N comprises L-arginine-¹⁵N₄.

7) The system according to claim 4, wherein said isotopically enriched molecular scaffold for said ¹⁹F comprises trimethoxy(3,3,3-trifluoropropyl)-silane.

8) The system according to claim 4, wherein said isotopically enriched molecular scaffold for said ^{79/81}Br comprises eosin-maleimide.

9) The system according to claim 4, wherein said isotopically enriched molecular scaffold for said ¹²⁷I comprises L-thyroxine.

10) The system according to claim 1, wherein a modified Stöber reaction is used to produce said silica nanoparticles having diameters in a range of 90 nm to 110 nm, wherein said modified Stöber reaction comprises a mixture of 100-nm silica nanoparticles comprising 0.7% (v/v) NH₃, 4% (v/v) of the silica precursor tetraethyl orthosilicate, and 0.31% (v/v) 3-mercaptopropyltrimethoxysilane (MPTMS) in 91% (v/v) aqueous isopropanol.

11) The system according to claim 1, wherein N-ethyl-d5-maleimide and eosin-maleimide were reacted with 3-mercaptopropyltrimethoxysilane (MPTMS) in dimethylsulfoxide (DMSO) under ambient conditions before said metalloid oxide silica nanoparticle were synthesized, wherein L-thyroxine was conjugated to said MPTMS using a heterobifunctional linker succinimidyl 4-N-maleimidomethyl-1-cyclohexane-carboxylate (SMCC) in a 1:1.1:1 ratio in DMSO for uniform and covalent incorporation within said silica nanoparticle matrix.

12) The system according to claim 1, wherein trimethoxy(3,3,3-trifluoropropyl)-silane and silane appended N-ethyl-d5-maleimide, eosin-maleimide and L-thyroxine are mixed into a Stöber reaction mixture in any (²H:¹⁹F:^{79/81}Br:¹²⁷I) isotope ratio to yield isotopically encoded silica nanotags.

13) The system according to claim 1, wherein trimethoxy(3,3,3-trifluoropropyl)-silane and silane appended N-ethyl-d5-maleimide, eosin-maleimide and L-thyroxine are mixed into Q Stöber reaction mixture in any 1:1:1:1 (²H:¹⁹F:^{79/81}Br:¹²⁷I) isotope ratio to yield isotopically encoded silica nanotags.

14) The system according to claim 1, wherein ionic metal isotopes are combinatorially mixed into a dispersion of silica nanoparticles to generate metal-based isotopically encoded silica nanotags.

15) The system according to claim 1, wherein said mass-based imaging platform is selected from the group consisting of multiplexed ion-beam imaging, and mass cytometry.

16) The system according to claim 1, wherein said elemental analysis platform is selected from the group consisting of X-ray fluorescence, energy dispersive X-ray spectroscopy, and laser induced breakdown spectroscopy.

17) The system according to claim 1, wherein a mixture of said isotopically encoded nanotags are applied to a substrate gold-coated silicon substrate for use in a multi-ion beam image.
